(19) United States
(12) Patent Application Publication
(10) Pub. No.: US 2008/0176269 A1
(43) Pub. Date: Jul. 24, 2008

(54) Method for determination of DHT levels in tissue samples

(76) Inventors: James L. Mohler, Buffalo, NY (US); Mark Titus, East Amherst, NY (US); Kenneth Tomer, Cary, NC (US)

Correspondence Address: HODGSON RUSS LLP;THE GUARANTY BUILDING, 140 PEARL STREET, SUITE 100, BUFFALO, NY, 14202-4040, US

(21) Appl. No.: 11/973,127

(22) Filed: Oct. 8, 2007

Related U.S. Application Data
(60) Provisional application No. 60/850,028, filed on Oct. 6, 2006.

(51) Int. Cl. C12Q 1/02; G01N 33/92

(52) U.S. Cl. 435/29; 436/71

(57) ABSTRACT

Provided is a method for determining the presence or amount of 5-α-dihydro-3-keto steroids (ADKSs) comprising the steps of preparing a sample for chromatographic separation, subjecting the sample to chromatographic separation, subjecting the chromatographically separated sample to atmospheric pressure photoionization (APPI), and detecting the amount of alkylated parent ions using mass spectrometry or methylated daughter ions by mass spectrometry after collisionally induced dissociation of the alkylated parent ions. Tandem mass spectrometry of ADKSs with a 6-member A ring and a 3-keto group using APPI and methanol as the alkoxy donor will produce methylated daughter ions with a mass/charge ratio of 85.0. Quantitation of this alkylated daughter ion provides a method to detect very low levels of ADKSs such as DHT. This method can also be used for the simultaneous detection of ADKSs and non-ADKSs.

This application claims priority to U.S. Provisional Application No. 60/850,028 filed on Oct. 6, 2006, the disclosure of which is incorporated herein by reference.

### FIELD OF THE INVENTION

This invention relates to the field of detection of steroids and more particularly provides a sensitive method for detection of 5-α-dihydro-3-keto steroids.

### BACKGROUND OF THE INVENTION

Identification and accurate quantification of steroid compounds involved in biological processes is relevant to diagnosis and development of therapeutic treatments of diseased conditions. For example, intracellular androgen quantification has become more important recently in some clinical situations. In androgen responsive tissues, intracellular testosterone (T) acts as a prohormone that is converted to dihydrotestosterone (DHT), a more potent androgen receptor ligand. In the prostate, the enzyme steroid 5α-reductase (SRD5A; EC 1.3.99.5) metabolizes T to DHT. SRD5A inhibitors, finasteride and dutasteride, have proven useful for treatment of benign prostate enlargement and are under investigation for prostate cancer prevention. It is also known that, prostate cancer recurs after androgen deprivation in almost all men and generally results in death despite castrate levels of circulating T. Continued investigation of new therapies for benign prostate enlargement, prostate cancer chemoprevention, and recurring prostate cancer require accurate measurement of DHT and other androgens in prostate tissue that can be obtained in small amounts via prostate biopsy. Detection and/or quantitation of prostate androgens is typically done using LC/ESI/MS/MS, where ESI is electrospray ionization. Attempts to apply the APPI method in the simultaneous detection and/or quantitation of prostate androgens, which offers the advantage of lower background signal and greater sensitivity for some steroids, has not heretofore been successful in part due to inability to detect DHT. Therefore, there is an ongoing need in the steroid area to develop a sensitive method for low-level, simultaneous detection of a wide range of steroids in biological and non-biological samples.

### SUMMARY OF THE INVENTION

The present invention provides a sensitive method for identification and quantification of 5-α-dihydro-3-keto steroids (ADKSs). The method permits simultaneous quantification of steroids at femtomole levels. Therefore, only a relatively small sample is required. The method is based on the generation of alkylated product ions of the ADKSs and their subsequent detection using mass spectroscopy. In one embodiment, this method can be used to detect and quantify androgen levels in prostate biopsy samples. In another embodiment, DHT is detected as methylated product ions. In another embodiment, DHT is quantitated as methylated daughter ions. Simultaneous detection of steroids, including ADKSs and non-ADKSs, can be carried out in any sample.

The present method includes the steps of providing a sample, subjecting the sample to chromatographic separation, subjecting the chromatographic eluent to atmospheric pressure photoionization (APPI) such that alkylated ADKS product ions are produced, and mass spectral analysis of the resulting ions. Each ADKS produces alkylated ADKS product ions having characteristic mass/charge ratio. Detection of the characteristic alkylated product ions (also referred to herein as “the parent ions”) is indicative of a particular ADKS in the sample.

Subjecting the alkylated parent ions (product ions from APPI) to collisionally induced dissociation (CID) in a tandem mass spectrometry (MS/MS) analysis results in formation of alkylated daughter ions. The production of m/z 85.0 alkylated daughter ions is common to all ADKSs when methanol is used as the dopant. Detection of alkylated daughter ions, coupled with identification of the characteristic alkylated parent ions, is indicative of the specific ADKS.

The present method can be used for detecting ADKSs, including DHT, in biological as well as non-biological samples. Therefore, this method can be used for detection of low levels of ADKSs for diagnostic or prognostic purposes. This method is also useful for identification or quantification of ADKSs in various manufacturing processes.

### BRIEF DESCRIPTION OF THE DRAWINGS

FIG. 1. Structures of proposed DHT parent ion adduct detected in quadrupole 1 (Q1) and daughter ion structure detected in quadrupole 3 (Q3).

FIG. 2. Reverse phase HPLC elution profile (ESI) of seven steroids (minutes); androstenedione (ASD) (26.1), 3β,17β-androstenediol (5-diol) (28.7), testosterone (T) (30.3), dehydroepiandrosterone (DHEA) (30.8), androstanedione (5α-ASD) (33.9), dihydrotestosterone (DHT) (36.2) and androsterone (AND) (38.8).

FIG. 3. Reverse phase HPLC elution profile (APPI) of seven steroids (minutes); androstenedione (ASD) (7.2), testosterone (T) (8.1), 3β,17β-androstenediol (5-diol) (8.1), dehydroepiandrosterone (DHEA) (8.8), dihydrotestosterone (DHT) (10.0), androsterone (AND) (11.3) and androstanedione (5α-ASD) (14.4).

FIG. 4. (A) Tandem mass spectrometry DHT chromatograms using photoionization source. DHT photoionization and scan using quadrupole 1. (B) Total ion chromatogram using 40 ng of DHT.

FIG. 5. Tandem mass spectrometry DHT product (parent) ions using ESI or APPI. (A) The DHT parent (product) ion m/z 291.2 forms the daughter ion m/z 255.2 using ESI source. (B) The methylated DHT parent (parent) ion m/z 305.0 forms daughter ion m/z 85.0 using APPI source and methanol dopant.

FIG. 6. Tandem mass spectrometry of alkylated DHT product (parent) ion using APPI with deuterated methanol (CD₃OH).

FIG. 7. Tandem mass spectrometry of alkylated DHT product (parent) ion using APPI with ethanol.

FIG. 8. DHT levels in castration-recurrent prostate cancer samples.

### DETAILED DESCRIPTION OF THE INVENTION

Subjecting steroids to APPI under standard conditions (using toluene and methanol), is known to produce molecular product ions with an m/z corresponding to M+H, as detected using mass spectroscopy, where M is the molecular mass of the molecule and H reflects the proton. For example, subjecting T to this method is known to yield a product ion having a m/z ratio of 289.2 corresponding to the addition of a proton to the T molecule to give rise to a (T+H)⁺ cation. Using the same reasoning, DHT can be expected to appear at a m/z ratio of 291.2—corresponding to DHT+H⁺. However, only a weak peak is observed at m/z ratio of 291.2 at high input concentrations. The absence of a peak at the expected location has resulted in investigators overlooking the utility of the APPI method in the detection of DHT.

In the present invention, it was surprisingly observed that for DHT and other 5-α-dihydro-3-keto steroids (ADKSs), the APPI method produces alkylated product ions rather than the conventional M+H ions. Thus, DHT, when subjected to APPI under standard conditions, does not produce the expected M+H cation (m/z 291.2), but instead produces a methylated product ion having a m/z of 305.0.

The present method is therefore based on the unexpected finding that alkylated ion species are produced using the APPI method. Such alkylated ion species can be detected and quantified using mass spectrometry. Each ADKS produces an alkylated product ion having a characteristic m/z ratio. Therefore, increased sensitivity of detection can be achieved by using atmospheric pressure photoionization (APPI) of chromatographically separated materials to produce characteristic alkylated product ions.

Mass spectrometry (MS) can be used to identify and quantify characteristic alkylated product (parent) ions produced during APPI. By using a combination of APPI and tandem mass spectrometry (MS/MS), the sensitivity of detection of ADKSs can be significantly increased over the conventionally used combination of ESI and MS/MS. In one embodiment, the characteristic parent ions generated by APPI are further subjected to collisionally induced dissociation (CID) to generate daughter ions, the presence and amount of which can be identified using further mass spectrometry in a MS/MS analysis.

A general chemical structure of ADKSs detectable by the present method is shown below in Formula 1. In Formula 1, R₁ can be H, ketone, acetate, hydroxyl, CH₃ or any alkyl group up to C₈H₁₇; R₂ can be CH₃, or other alkyl groups and R₃ can be CH₃ or other alkyl groups. In one embodiment R₁ is OH, R₂ is CH₃ and R₃ is CH₃, which corresponds to DHT. In another embodiment, R₁ is double bond 0 (ketone group), R₂ is CH₃ and R₃ is CH₃, which corresponds to androstanedione (5α-ASD). In another embodiment, R₁ is C(O)CH₃ (acetate group) and R₂ is CH₃ and R₃ is CH₃, which corresponds to dihydroprogesterone (DHP). In another embodiment R₁ is C₈H₁₇, R₂ is CH₃ and R₃ is CH₃, which corresponds to cholestanone.

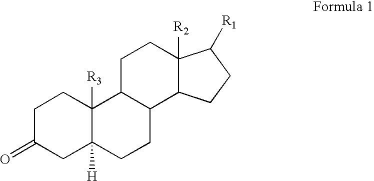

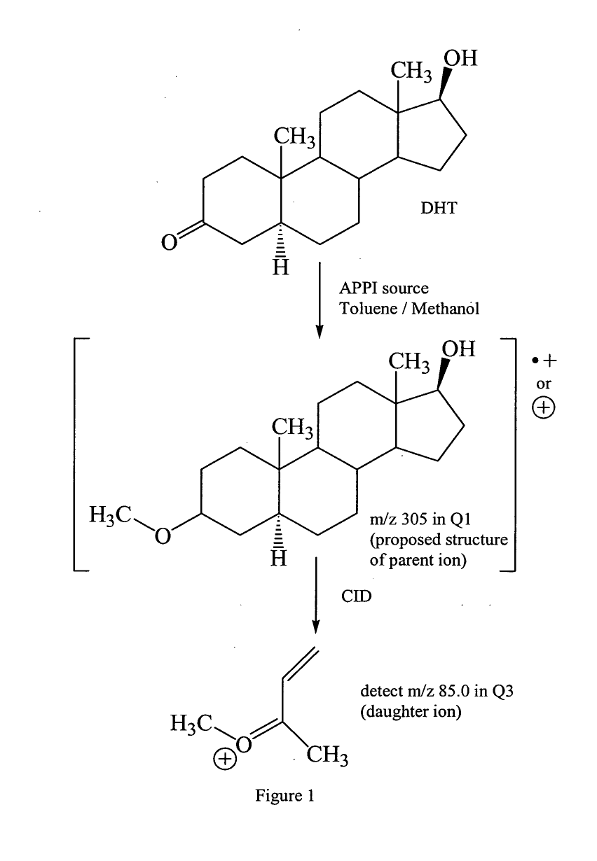

Using APPI ionization, an ADKS can be detected by MS or MS/MS as an alkylated adduct. In the APPI ionization step, a sample suspected of containing the ADKS is subjected to ultraviolet radiation in the presence of a dopant, such as but not limited to toluene, benzene, anisole, and acetone, and an alkoxy donor, such as but not limited to methanol, ethanol or tertiary butanol, resulting in formation of an ether functional group at carbon 3 (see Formula 1). Each ADKS will produce a characteristic methylated product ion (e.g., methyl ether at carbon 3 in Formula 1) using methanol in mobile phase. For DHT the methylated parent ion has a m/z 305.0. Although not intending to be bound by any particular theory, a schematic representation of the contemplated formation of the DHT methylated product ion is depicted in FIG. 1. The methylated parent ions for 5α-ASD, DHP, and cholestanone have m/z 303.2, 331.3, 402.4, respectively. It should be noted that based on our experiments, it is considered that a steroid must have a 6-member A ring and a 3-keto group in order for a steroid to form a alkylated product ion under the APPI conditions described herein. This is supported by the inability of T or other Δ4-3-keto steroids to produce alkylated product ions detectable under our experimental conditions.

While the characteristic alkylated parent ions can be identified using MS, a further step of subjecting the characteristic alkylated parent ions to tandem mass spectrometry analysis, where CID leads to production of characteristic daughter ions, which are detected as a peak corresponding to ions with a m/z of 85.0 when methanol is used as the alkoxy donor, provides a further sensitive method for detection of ADKSs. Steroids with a 6-member A ring, a 3-keto group, and a 5-α hydrogen (trans A-B ring junction) will give an alkylated daughter ion in the APPI/MS/MS analysis (see FIG. 1). If the identity of the parent ions that give rise to the alkylated daughter ions is known, the amount of the ADKS in the original sample can be calculated. Each ADKS has a unique characteristic chromatographic retention time, based on the particular chromatographic conditions used. An alkylated parent ion is characterized by an individual chromatographic retention time-specific to a particular ADKS under a given set of chromatographic conditions. Each parent (or product) ion also has a characteristic mass/charge ratio. Thus, alkylated daughter ions are characteristic of a specific ADKS because the alkylated parent ions from which they are formed can be identified based on their mass/charge ratio and chromatographic retention time. For example, if after APPI using methanol as alkoxy donor and MS analysis, the parent methylated ion peak is observed at m/z 305 (indicative of the presence of DHT in the test sample), and after CID and MS analysis, the methylated daughter ion is observed at m/z 85.0, the amount of DHT in the test sample can be determined. Thus, when analyzing a sample of prostate tissue using LC/APPI/MS/MS this methylated daughter ion can be used as an indicator of prostate DHT levels.

Sample requirements for detection and quantitation of an ADKS using the present method are minimal. For this reason, the present method is useful particularly for tissues from which biopsy samples are typically difficult to obtain. For example, biopsy samples obtained from tissues such as prostate and other solid organs are not typically amenable to meaningful analysis due to insufficiency of the analyte material in sample. However, by increasing the sensitivity of detection ADKSs, DHT for example, biopsy samples containing only limited quantities of ADKSs can be analyzed without resorting to surgical sampling. Therefore, the present method provides an improvement over RIA or LC/ESI/MS/MS (Mohler, J. L, et. al. Clin Cancer Res 2004, 10, 440-8, Titus M A, et. al. Clin Cancer Res 2005, 11, 4653-7) for the quantitation of a broad range of steroids.

The method of the invention is considered to be particularly useful in diagnosing prostate conditions. A prostate tissue sample can be obtained using routine prostate biopsy procedures. Generally, collection from multiple biopsy sites is preferable to maximize diagnostic sensitivity. The ADKSs can be extracted from the tissue using standard methods. For example, protease treatment followed by solid phase extraction can be used. Internal standards, e.g. deuterated ADKS (such as 0.5 ng), can be added to each sample. Subsequent analysis of the sample by LC/MS/MS using APPI results in sensitive detection of multiple steroids including ADKSs, such as DHT and 5α-ASD, in the sample which can be used in diagnosis and development of therapeutic treatments for prostate cancer.

The present method generally involves the steps of providing a sample, subjecting the sample to chromatographic separation, subjecting the chromatographically separated sample to APPI in the presence of a dopant and an alkoxy donor resulting in formation of alkylated (such as methylated) parent ions, and performing MS analysis of the parent ions to identify and quantify the alkylated parent ions. In one embodiment, the methylated parent ions are further subjected to CID and MS analysis to generate, identify and quantify m/z 85.0 daughter ions. While the production of alkylated product ions can suffice for the identification and quantification of the ADKS, generation of alkylated daughter ions provides further confirmation of the presence of the ADKS (by eliminating interference from any overlapping peaks in the MS data), as well as enables accurate quantitation of the ADKS. An additional advantage is the uniqueness of the parent/fragment (or daughter) pairs produced by APPI of ADKS. For example in the ESI/MS/MS analysis of DHT, the pair is m/z 291.2/255.2. The m/z 255.2 ion is consistent with the loss of two water molecules from the DHT molecular ion (m/z 291.2). There are many structures which could have this mass consistent with the loss of two water molecules from the DHT molecular ion. The m/z 305.0/255.0 and m/z 305.0/85.0 pairs seen in APPI are unique, reducing background and boosting confidence in species assignment.

Suitable test samples include any sample that can contain ADKSs. The samples may be biological samples or non-biological samples. Non-biological samples can include those generated in manufacturing or analytical lab work, or environmental samples. Biological samples may be obtained from any biological source, such as an animal (including humans and non-humans), cell culture, organ culture, etc. Suitable biological samples include blood, plasma, serum, hair, muscle, urine, saliva, tear, cerebrospinal fluid, or other tissue sample. Such samples may be obtained, for example, from a patient presenting himself in a clinical setting for diagnosis, prognosis, or treatment of a disease or condition. Test samples may also be non-biological such as those obtained in manufacturing or analytical settings.

Separation of ADKS from the Test Sample.

Separation of ADKSs and non-ADKSs in the test sample provides the ADKSs and non-ADKSs in a form that can be ionized and the ionized product(s) subjected to mass spectral analysis. For example, ADKSs and non-ADKSs can be chromatographically separated such as by using high-performance liquid chromatography (HPLC). For example, reverse-phase HPLC columns can been used to separate ADKS and non-ADKS. Chromatographic separation provides ADKSs and non-ADKSs in a form amenable to subsequent ionization and mass spectral analysis. The term “chromatographically separated” or “chromatographic separation” as used herein refers to the process of subjecting a sample to the chromatographic separation procedure. It is not necessary to collect separate fractions. Rather, ADKSs and non-ADKSs can be identified by using analytical methods in a continuous manner on the eluent from the column. Similarly, “chromatographically separated ADKSs” and “chromatographically separated non-ADKSs” refer to the ADKSs and non-ADKSs present in the eluent from the chromatographic column, which can be identified according to their temporally-based position in the eluent, i.e. retention time. Other separation techniques could be used, capillary electrophoresis for example.

### Ionization of Chromatographically Separated ADKS Resulting in Formation of Alkylated Product Ion.

The sample containing chromatographically separated ADKSs and non-ADKSs is subjected to an ionization process which generates an alkylated ADKS ion, i.e. a charged form of the alkylated ADKS molecule, and non-ADKS ions. This method utilizes APPI whereby a molecule (e.g., an ADKS) is subjected to ultraviolet radiation, in the presence of a dopant, e.g. toluene, and an alkoxy donor, e.g. methanol and ethanol. The result of such APPI ionization of ADKSs is formation of a positively-charged, alkylated form of the ADKS molecule, referred to as an alkylated product ADKS ion (or alkylated parent ADKS ion in the tandem mass spectrometry analysis). It will be recognized by those skilled in the art that not all ADKS molecules or non-ADKS molecules will be ionized in the APPI process for the sensitive detection of ADKSs and non-ADKSs in a sample. FIG. 1 depicts a schematic representation of the chemical structures involved in formation of the methylated DHT parent (m/z 305.0) and daughter ion (m/z 85.0) in a tandem mass spectrometry experiment.

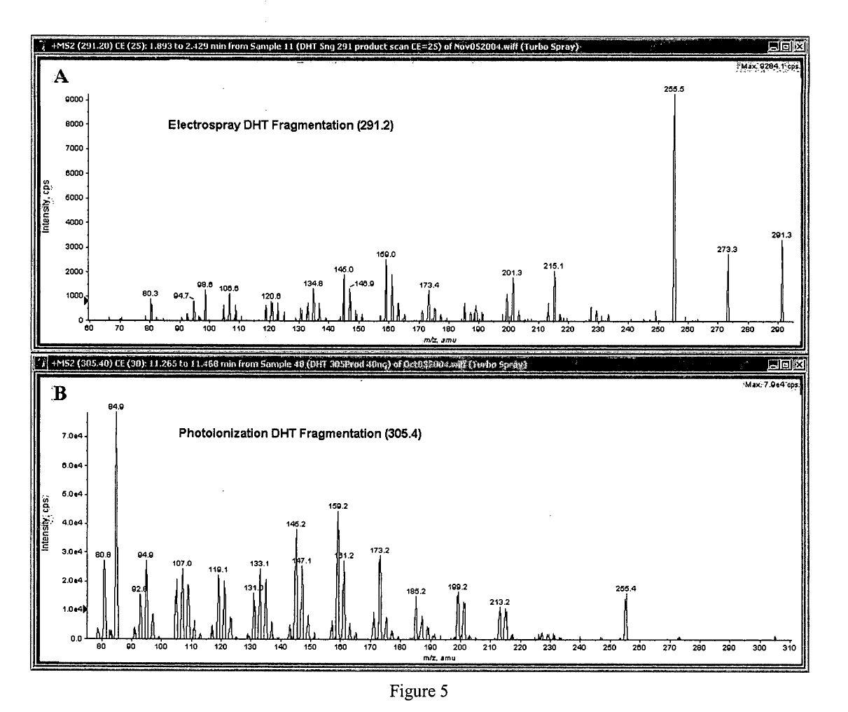

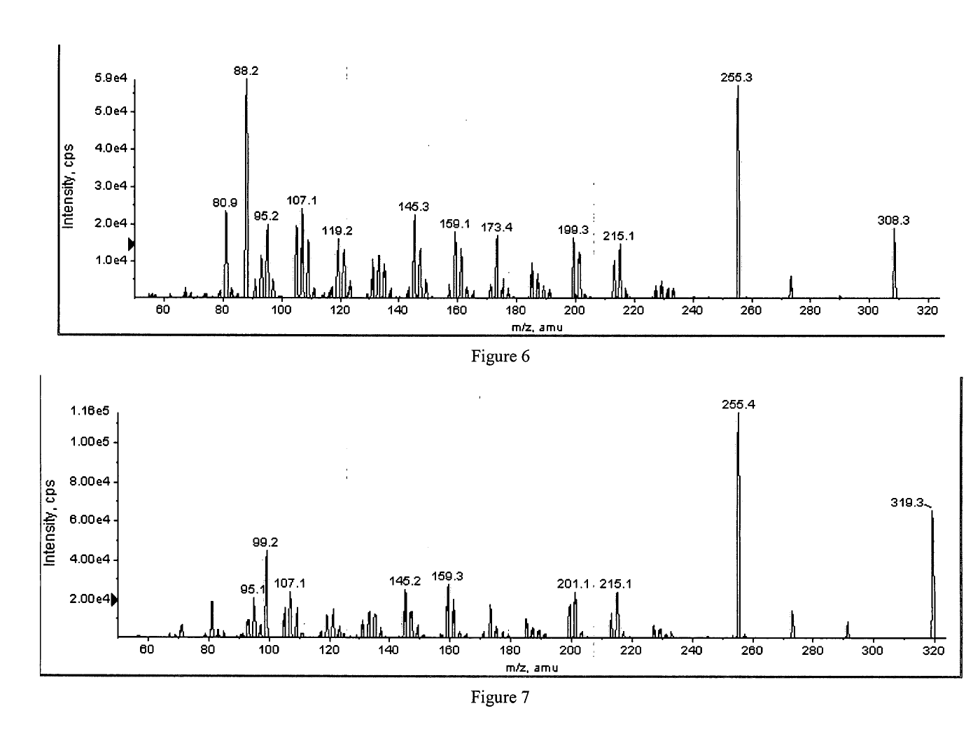

When APPI is carried out in the presence of methanol, it is believed that methanol acts as a methoxy donor in the formation of methylated ADKS product ions. The methylated ADKS product ion has a m/z ratio corresponding to the addition of 15 mass units (Daltons) to the ADKS molecule when standard HPLC grade methanol is used as the methoxy donor. Methanol is considered to be the source (donor) of the methyl group based on our studies with deuterated methanol. When deuterated methanol (CD₃OH) is used in the APPI process the alkylated ADKS product ion has a m/z three Daltons greater than that observed using methanol. For example, when DHT was subjected to APPI in the presence of deuterated methanol the deuterated product ion was detected at a m/z of 308.0 and the deuterated daughter ion was detected at a m/z 88.0 (see FIG. 5A). The mass to charge ratios of both the product and daughter ions in the case of APPI using deuterated methanol are each three Daltons greater, indicating formation of deuterated ions, than those detected in the absence of deuterated methanol. Additionally, when APPI was performed in the presence of ethanol the ethyl ether was observed at a m/z 319.3, which is 14 Daltons greater than the parent ion observed in the presence of methanol, corresponding to the additional CH₂. The fragment at m/z 85 also shifted 14 Daltons to 85.0 (see FIG. 5B). These results support the conclusion that small molecular weight alcohols, e.g. methanol, ethanol, and tertiary butyl alcohol, act as alkoxy donors in the APPI process. In one embodiment, DHT is quantified using the amounts of both the m/z 255.0 daughter ion and m/z 85.0 methylated daughter ion.

### Mass Spectral Analysis of ADKS Ions (MS, MS/MS).

“Mass spectrometry” or “MS” as used herein refers to methods of filtering, detecting, and measuring ions based on their mass/charge ratio, or m/z. In general, one or more molecules of interest are ionized, and the resulting ions introduced into a mass spectrometric instrument where, due to a combination of electric and magnetic fields, the ions follow a path in space that is dependent on the ion's mass (m) and charge (z).

The mass spectrometer can take a variety of forms. Examples of MS instruments include instruments based on quadrupole or quadrupole ion trap architectures and time of flight instruments. In the quadrupole or quadrupole ion trap instruments, ions in an oscillating radio frequency field experience a force proportional to the DC potential applied between electrodes, the amplitude of the RF signal, and charge on the ions. The voltage and amplitude can be selected so that only ions having a particular m/z travel the length of the quadrupole.

The sensitivity of a mass spectrometry experiment can be enhanced by using tandem mass spectrometry or MS/MS. In tandem mass spectrometry a precursor ion or group of ions generated from a molecule (or molecules) of interest may by filtered in an MS instrument, and these precursor ions (or parent ions) subsequently fragmented to yield one or more fragment ions (or daughter ions) that are then subjected to a second MS analysis. By careful selection of precursor (parent) ions, only ions produced by analytes of interest are passed to the fragmentation chamber, where collision with atoms of inert gas occurs to produce fragment ions. Because both the precursor and fragment ions are generated in a reproducible fashion under a given set of ionization/fragmentation conditions, the MS/MS technique can provide an extremely powerful analytical tool to identify and quantify molecules of interest. For example, the combination of filtration can be used to eliminate interfering substances, and can be particularly useful in complex samples, such as biological samples.

In one embodiment a triple quadrupole instrument in used to perform the MS/MS analysis. In a triple quadrupole mass spectrometry instrument three quadrupole mass analyzers are used linearly. The first (Q1) and third (Q3) quadrupoles act as mass filters, and the second quadrupole (Q2) is employed as a collision cell. This collision cell is not used as a mass filter but only as a means to induce collisional dissociation of selected parent ion(s) from the first quadrupole. Subsequent fragments from the collisional cell are passed through to the third quadrupole where they may be filtered or scanned fully to give the mass/charge ratio of the daughter ions produced in the collision cell.

Quantitation of unknown amounts of atoms or molecules (analytes) in test samples by mass spectrometry is well known by those skilled in the art. The ion signal of a compound generated by a mass spectrometer is proportional to the amount of the analyte used to generate that signal. For example, a known amount of DHT will give rise to a proportional daughter ion signal (m/z 85.0). Based on this proportionality between ion signal and amount of analyte, the amount of an analyte in a sample can be determined by correlating the ion signal of an unknown amount of an analyte to the ion signal generated by a known amount (mass) of standard. The standard can be a pure form of the analyte or an isotopically labeled form of the analyte, such as a deuterated form of the analyte where deuterium atoms have been incorporated into the analyte in place of some or all of the hydrogen atoms. The ion signal generated by the standard can be obtained by running the sample independently from the test sample. This is commonly referred to as use of an external standard. The standard can also be run at the same time as the test sample by adding, commonly referred to as spiking, for example, a deuterated analog of the analyte of interest to the test sample which is referred to as use of an internal standard.

In one embodiment, the daughter ion signal (m/z 85.0) generated by an unknown amount of DHT in a test sample is correlated to the daughter ion signal (m/z 85.0) generated by standard samples of known quantities of DHT run independently from the test sample. Using the proportional relationship of the daughter ion signal to mass of DHT giving rise to that daughter ion signal, the amount of DHT in the test sample can be determined. In another embodiment, a known amount of deuterated ADKS is added, or spiked, to the test sample and the ion signals obtained for the known amount of deuterated ADKS standard and unknown amount of non-deuterated ADKS. Based on the proportionality of amount (mass) of ADKS to ion signal, the ion signal of an unknown amount of ADKS can be correlated to the ion signal of the deuterated ADKS standard and the amount of ADKS in the test sample determined.

Utilizing the identification and low-level quantitation of ADKSs such as DHT and 5α-ASD in prostate samples as a diagnostic tool to address prostate cancer diagnosis and treatment is a very useful application of this method. Examples of other uses of this method include quantitation of: ADKSs in samples acquired from men taking testosterone replacement, DHT levels in scalp samples, DHT levels in plasma from men on androgen deprivation therapies, phase 2 metabolites of DHT levels in urine samples (after treatment of sample with B-glucuronidase).

This invention is further described through the following examples, which are to be construed as illustrative and not restrictive in any way.

### EXAMPLE 1

This example describes the quantitation of seven steroids, including ADKSs, by both LC/ESI/MS/MS and LC/APPI/MS/MS.

Chemicals and Reagents. Deuterated T and DHT internal standards were purchased from CDN Isotopes (Pointe-Claire, Quebec, Canada). T, DHT, DHEA, ASD, 5α-ASD, AND and 5-diol standards were purchased from Steraloids (Newport, R.I.). Acetic acid (Sigma-Aldrich, St. Louis, Mo.) was used as mobile phase modifier and toluene (J. T. Baker) was used as the photoionization dopant. Hexane, ethyl acetate and methanol were purchased from J. T. Baker. All Chemicals used were of analytical or chromatographic grade. Water was purified using the Milli-Q Water Purification System (Millipore, Molsheim, France).

Analytical Instrumentation. Liquid chromatography tandem mass spectrometry analyses of androgens were conducted using an Agilent 1100 capillary LC system (Palo Alto, Calif.), coupled to an Applied Biosystems/MDS Sciex API-3000 triple quadrupole mass spectrometer (MDS Sciex, Concord, ON, Canada). Atmospheric pressure photoionization (APPI) analysis incorporated a PhotoSpray source (Applied Biosystems, Foster City, Calif.). ESI analyses used a TurboSpray source (Applied Biosystems). APPI analysis used a Luna C18(2) 5 mm 150×2 mm column (Phenomenex, Torrance, Calif.) and ESI analysis used a Zorbax SB-C18 5 mm 150×0.5 mm column (Agilent) chromatography columns. SPEC C18AR 15 mg, 3 mL columns (Varian, Palo Alto, Calif.) were used for solid phase extraction (SPE). The data were collected and processed using PE Sciex Analyst 1.2 software.

HPLC Separation. Stock mixtures containing each of the seven steroids and two deuterated androgens at 0.1 mM were prepared and diluted with ethanol to appropriate concentrations for calibration curves and mobile phase optimization for baseline separation of seven steroids.
ESI Method (T, DHT, DHEA, ASD, AND, 5α-ASD and 5-diol). An Agilent 1100 capillary LC system consisting of a G1376A CapPump equipped with a vacuum solvent degasser, a G1377A μ-WPS autosampler and a G1330B ALS Therm column compartment containing a Zorbax SB-C18 (5 um, 150×0.5 mm, Agilent) column was used to separate T, DHT, DHEA, ASD, 5α-ASD, AND and 5-diol. The column was maintained at 60° C. and flow rate of 20 μL/min. A gradient profile using mobile phase A (0.1% acetic acid in water) and B (0.1% acetic acid in methanol) at flow rate 20 μL/min was used as follows: 40% to 53% B from 0.0-26.0 min., 54% to 100% B from 26.1-41.7 min., 100% B from 41.8-47.0 min., and 100-40% B from 47.0-49.0 min. The column was equilibrated at 40% B for 10 minutes prior to sample injection. After analysis of each prostate sample, a blank injection washed the column and a third injection re-equilibrated the column prior to next sample injection.
APPI Method (T, DHT, DHEA, ASD, AND, 5α-ASD and 5-diol). The LC system and assay was the same as described above with minor modifications. The aqueous phase was 2 mM ammonium formate (pH 3.1) and organic phase was 2 mM ammonium formate (pH 3.1) in methanol. A reverse-phase, Phenomenex Luna C18, 3 um, 150×2.00 mm, column was operated at 175 μL/min using injection volume 5 μL. The column was maintained at 60° C.
Mass Spectrometry. The column eluate was split at a ratio of 1:10 before sample was introduced to the mass spectrometer. All prostate specimen samples were randomized and tandem MS/MS was performed on 14 consecutive days.
ESI Method. T, DHT, DHEA, ASD, 5α-ASD, AND, and 5-diol were detected using positive multiple reaction monitoring mode (+MRM). ASD was monitored at m/z transition 287.2>97.0. DHT, AND, and 5-diol were monitored at m/z transition of 291.2>255.2 (−2H₂O). T and 5α-ASD were monitored at m/z transition 289.2>97.0. DHEA was monitored at m/z transition 289.2>213.2. Dwell times were varied so that a minimum of 16 data points were collected across the chromatographic peaks. T here was a 5 ms pause between MRM acquisitions. Quadrupole 1 was set to unit resolution and quadrupole 3 was set to low resolution. Internal standards DHT-d₃ and T-d₃ were monitored at m/z 294.2>258.2 and 292.2>97.0, respectively (Table 1). Nitrogen was used for all gas inputs. The turbo gas was set to 6 L/min and the source was offset to 4 horizontally and 4 laterally. Detection settings for all androgens were: nebulizer gas pressure 6 psi, curtain gas flow rate 12 L/min, ion spray voltage 3900 and turbo gas temperature 300° C.
APPI Method. Toluene was introduced to the photospray source using a syringe pump or LC pump with restrictive capillary at a flow rate 20 μL/min. The source temperature was set to 350° C. Nitrogen was used for all gas inputs. The UV lamp gas flow was 1 L/min. The auxiliary gas for toluene delivery to ion source was set at 9.0. The nebulizer gas was introduced via a regulator set to 75 psi. Dwell times were varied so that a minimum of 16 data points were collected across the chromatographic peaks with a 5 ms pause between MRM acquisitions. Quadrupole 1 was set to unit resolution and Quadrupole 3 was set to low resolution. Table 2 lists remaining instrumental settings. Instrument settings for T and DHT detection were: auxiliary (nebulizer) gas pressure 9, curtain gas flow rate 10, ion spray voltage 1300 V and gas temperature 300° C.

TABLE 1
ESI multiple reaction monitoring m/z transitions and individual
optimized operating parameters in volts: declustering potential
(DP), focusing potential (FP), entrance potential (EP), collision
energy (CE) and collision cell exit potential (CXP).

| Androgens | m/z Transitions | DP | FP | EP | CE | CXP |
|---|---|---|---|---|---|---|
| ASD | 287.2 > 97.0 | 28 | 85 | 6 | 32 | 8 |
| DHT, AND, 5-diol | 291.2 > 255.2 | 37 | 105 | 6 | 22 | 6 |
| T, 5α-ASD | 289.2 > 97.0 | 36 | 105 | 7 | 32 | 6 |
| DHEA | 289.2 > 213.2 | 24 | 80 | 5 | 24 | 6 |
| T-d₃ | 292.2 > 97.0 | 36 | 105 | 7 | 32 | 6 |
| DHT-d₃ | 294.2 > 258.2 | 37 | 105 | 6 | 22 | 6 |

TABLE 2
APPI multiple reaction monitoring m/z transitions and individual
optimized operating parameters in volts: declustering potential
(DP), focusing potential (FP), entrance potential (EP), collision
energy (CE) and collision cell exit potential (CXP).

| Androgens | m/z Transitions | DP | FP | EP | CE | CXP |
|---|---|---|---|---|---|---|
| ASD | 287.2 > 97.0, | 28 | 115 | 10.5 | 32.5, | 5, |
| | 109.0 | | | | 34 | 6 |
| T | 287.2 > 97.0, | 28 | 115 | 10.5 | 33, | 5, |
| | 109.0 | | | | 35 | 6 |
| DHT | 305.2 > 255.2, | 29 | 135 | 10.5 | 24, | 5.7, |
| | 85.0 | | | | 39.6 | 4.5 |
| 5-diol | 273.2 > 255.2, | 20, | 100 | 10.5 | 21, | 5.5, |
| | 255.2 > 159.1 | 25 | | | 27 | 9.5 |
| 5α-ASD | 303.2 > 253.1, | 26 | 120 | 10.5 | 25, | 5.7, |
| | 85.0 | | | | 37 | 4.5 |
| DHEA | 271.2 > 213.2, | 30 | 130 | 10.5 | 22.5, | 4.5, |
| | 91.2 | | | | 58 | 9 |
| T-d₃ | 292.2 > 97.0 | 28 | 115 | 10.5 | 33 | 5 |
| DHT-d₃ | 308.2 > 258.2 | 29 | 135 | 10.5 | 24 | 5.7 |

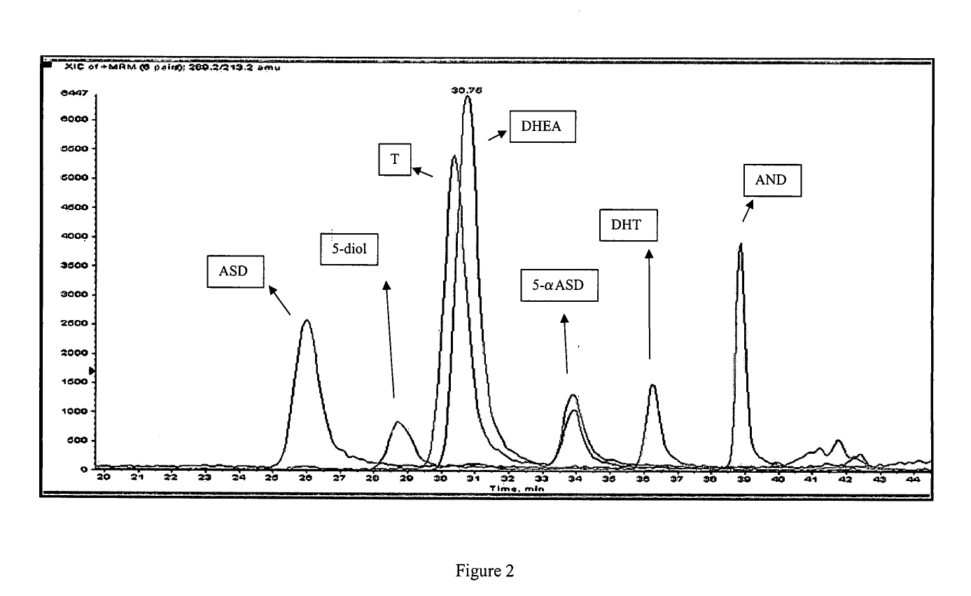

Patient and Tissue Samples. Prostate specimens were immediately frozen in liquid nitrogen within 5 minutes of procurement to prevent ischemic damage. Recurrent prostate cancer tissue was obtained from men who underwent transurethral resection to relieve urinary retention from local recurrence during androgen deprivation therapy. Androgen-stimulated benign prostate specimens were obtained from radical prostatectomy specimens from men with clinically localized prostate cancer. The superior vascular pedicles were left intact until all other portions of the procedure were completed. Upon removal of the operative specimen, the surgeon took the prostate to a side table, the specimen was inked and samples were obtained and frozen immediately in liquid nitrogen. Histological diagnoses were confirmed by examination of frozen and corresponding formalin-fixed, paraffin-embedded tissue specimens. Data from research activities was merged with matched clinical data from prospectively maintained databases. All prostate specimens were acquired in compliance with the guidelines of the University of North Carolina at Chapel Hill Lineberger Comprehensive Cancer Center Clinical Protocol Review Committee and Institutional Review Board and the federal Health Insurance Portability Accountability Act (HIPAA) and protected health information (PHI) regulations.
Tissue Extraction. Cryopreserved prostate specimens were homogenized at 50 mg/mL in 4° C. purified double distilled water. One mL aliquots were removed and 1 ng each of deuterated T and DHT internal standards were added and samples extracted 3 times with 3 ml 60:40 hexane:ethyl acetate. Combined organics were washed once with 0.5 mL 5% NaHCO₃ and evaporated under vacuum. Analyte purity, sample concentration and matrix effects were improved using solid phase extraction cartridges (SPEC-C18AR, Varian). Solid phase extraction cartridges were conditioned with methanol, then water and samples were applied in 1:5 methanol:water. Cartridges were washed with purified distilled water and 20% methanol in water. A vacuum of ˜12 mm Hg was applied for 5 minutes to dry the cartridges. Samples were eluted in methanol, dried under vacuum, and reconstituted in 30% methanol for analysis.
Results. The seven C19 steroids were isolated from prostate tissue homogenates containing isotopically labeled internal standard, T-d₃ and DHT-d₃, using liquid-liquid extraction and purified using C18 solid-phase extraction cartridges. The concentrated androgen mixture was reconstituted and separated using HPLC. Six of seven steroids standards were separated to baseline using a C18 capillary column and methanol/water gradient (FIG. 2). Retention times (minutes) were 26.1 for ASD, 28.7 for 5-diol, 30.3 for T, 33.9 for 5α-ASD, 36.2 for DHT, and 38.8 for AND. DHEA eluted at 30.8 minutes and was not completely resolved from T. Ionization was performed using ESI or atmospheric pressure photoionization APPI. Mass transitions were monitored at 287.2>97.0 m/z for ASD, 289.2>97.0 m/z for T and 5α-ASD, 289.2>213.2 m/z for DHEA, 291.2>255.2 m/z for DHT, AND and 5-diol, 292.2>97 m/z for T-d₃ and at 294.2>258.2 m/z for DHT-d₃ in the positive multiple-reaction monitoring mode. The unique product ions for T (97) and DHEA (213.2) were used to resolve HPLC peaks and allow specific and sensitive quantitation.

Calibration curve correlation coefficients for the seven C19 steroids range from 0.9984 to 0.9999. Detection limits for T and DHT were 0.7 fmol or 203 fg using ESI and 0.15 fmol or 43 fg using APPI (signal-to-noise ratio of 3). The detection limits for DHEA, AND, ASD and 5α-ASD ranged from 1.0 to 8.6 fmol and 165 pmol for 5-diol using ESI (signal-to-noise ratio from 3 to 10). Validation data confirmed calibration of T and DHT within the linear dynamic range 0.7 fmol to 3.5 pmol and DHEA, AND, ASD, 5α-ASD and 5-diol within the linear dynamic range 3.5 fmol to 3.7 pmol. Limit of quantitation was 1.0 fmol to 3.5 pmol where accuracy remained ±12% for ESI and APPI. LC/APPI/MS/MS allows sensitive and reproducible analysis of T and DHT in prostate specimens. This method can be used to establish DHT metabolic and degradative pathways in prostate tissue and determine the origin of DHT in recurrent prostate cancer and prostate cancer tissue samples.

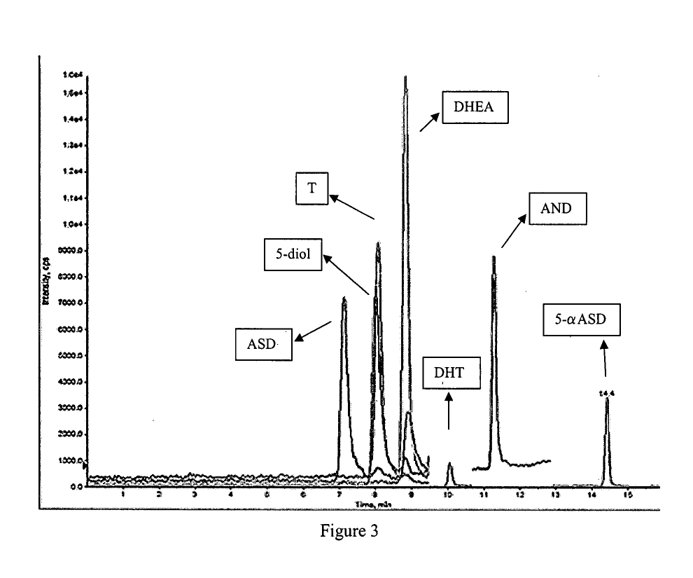

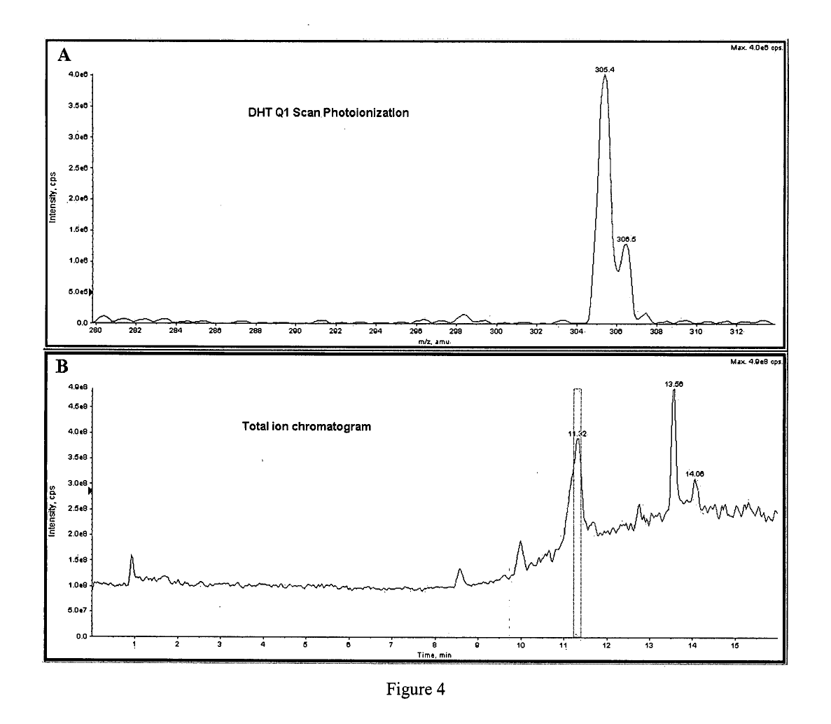

The same C19 steroid mass transitions were observed using the APPI source, however the DHT mass transition, m/z 291.2>255.2, was significantly decreased. To determine if DHT was forming an ion other than the protonated molecular ion, 40 ng DHT was ionized in the APPI source under isocratic LC conditions of 9:1 Methanol:Water, 0.1% acetic acid. Quadrupole 1 was scanned for molecular ions between m/z 280 and m/z 315. An intense peak at m/z 305 and its 13C satellite peak at m/z 306 were observed (FIG. 3A) using optimized photoionization conditions. This unique molecular ion corresponded to the peak at 11.32 minutes in the total ion chromatogram (FIG. 3B).

The product ion at m/z 305 corresponds to addition of 15 mass units to the DHT molecule. The predicted mechanism of methylation may involve acid catalyzed nucleophilic substitution of methanol at either carbon 3 or carbon 17 and loss of a single water molecule.

An intense product ion was observed (m/z 85.0) in quadrupole 3 after collision induced dissociation of the methylated DHT molecular ion. The predicted product ion structure detected in quadrupole 3 contains the unique methyl ether functional group (FIG. 1). The different DHT product ions detected using ESI or APPI are shown in (FIG. 5). The DHT molecular ion produced at m/z 291.2 using ESI shows a strong product ion at m/z 255.2 resulting from loss of 2 water molecules (FIG. 5A). DHT ionization using APPI shows a product ion at m/z 255.2 supporting methylation reactions at either the 3-keto or 17β-hydroxy functional groups (FIG. 5B). The intense peak at m/z 85.0 using APPI suggests this product ion is stable and reliable for DHT quantification in prostate cancer samples.

### EXAMPLE 2

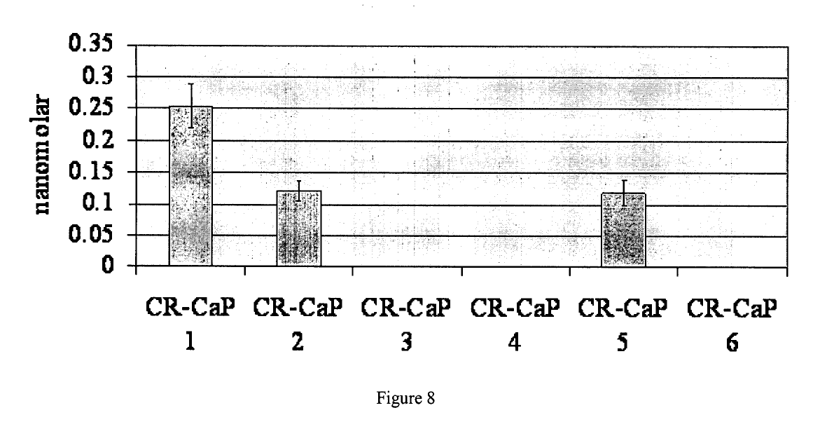

This example describes the determination of DHT levels in prostate samples using HPLC/APPI/MS/MS. Samples were taken from surgically harvested tissue and the DHT levels determined according to the method described in Example 1. The results are shown in FIG. 8.

FIG. 8 shows DHT levels in tissue samples taken from patients who have experienced prostate cancer recurrence after undergoing castration as part of an androgen deprivation prostate cancer treatment. These data demonstrate that DHT can be detected at femtomolar concentrations.

We claim:

1. A method of simultaneous detection of steroids in a test sample said steroids comprising non-5-α-dihydro-3-keto steroids (non-ADKSs) and 5-α-dihydro-3-keto steroids (ADKSs) comprising the steps of:
(a) subjecting the test sample to chromatographic separation;
(b) subjecting the chromatographically separated sample to atmospheric pressure photoionization (APPI) such that characteristic non-ADKS ions and characteristic alkylated ADKS ions are produced;
(c) detecting the presence or amount of non-ADKSs ions and characteristic alkylated ADKS ions using mass spectrometry.

2. The method of claim 1, wherein the chromatographic separation in step (a) is performed by high-performance liquid chromatography (HPLC).

3. A method of claim 1, where said APPI ionization is performed in the presence of a dopant and alkoxy donor, wherein the dopant is selected from a group consisting of toluene, benzene, anisole and acetone, and the alkoxy donor is selected from the group consisting of methanol, ethanol and tertiary butanol.

4. The method of claim 1, wherein the presence or amount of ADKS in the test sample is determined by comparison of the amount of characteristic alkylated ADKS ions to ions produced by a reference sample.

5. The method of claim 1 wherein the ADKS is selected from a group consisting of dihydrotestosterone (DHT), dihydroprogesterone (DHP), androstanedione (5α-ASD), and cholestanone.

6. The method of claim 5, wherein the ADKS is DHT and the characteristic alkylated ADKS ion is a methylated ion having a mass/charge ratio of 305.0.

7. The method of claim 1, wherein the test sample is a biological sample.

8. The method of claim 1, wherein the test sample is obtained from a human or non-human animal.

9. The method of claim 8, wherein the test sample is obtained by biopsy.

10. The method of claim 8, wherein the test sample is blood, plasma, serum, hair, muscle, urine, saliva, tear, or cerebrospinal fluid.

11. A method for determining the amount of 5-α-dihydro-3-keto steroids (ADKSs) in a sample comprising the steps of:
(a) subjecting the sample to chromatographic separation;
(b) subjecting the chromatographically separated ADKSs to ionization by atmospheric pressure photoionization (APPI) such that characteristic alkylated ADKS parent ions having a mass/charge ratio specific to the ADKS are produced;
(d) subjecting the methylated ADKS parent ions to collisionally induced dissociation to produce methylated daughter ions having a mass/charge ratio of 85.0; and
(e) determining the amount of ADKS in the sample by correlating the amount of said daughter ions to the amount of ADKS in the sample.

12. The method of claim 11, wherein the chromatographic separation in step (a) is performed using high-performance liquid chromatography (HPLC).

13. A method of claim 11, where said APPI ionization is performed in the presence of a dopant and alkoxy donor wherein, the dopant is selected from a group consisting of toluene and benzene, anisole and acetone, and the alkoxy donor is selected from the group consisting of methanol, ethanol and tertiary butanol.

14. The method of claim 11, wherein the presence or amount of ADKS in the test sample is determined by comparison of the amount of characteristic alkylated ADKS ions to ions produced by a reference sample.

15. The method of claim 11, wherein the ADKS is selected from a group consisting of dihydrotestosterone (DHT), dihydroprogesterone (DHP), androstanedione (5α-ASD), and cholestanone.

16. The method of claim 11, wherein the test sample is a biological sample.

17. The method of claim 11, wherein the test sample is obtained from a human or non-human animal.

18. The method of claim 17, wherein the test sample is obtained by biopsy.

19. The method of claim 17, wherein the test sample is blood, plasma, serum, hair, muscle, urine, saliva, tear, or cerebrospinal fluid.
